(19) United States
(12) Patent Application Publication
(10) Pub. No.: US 2005/0227966 A1
(43) Pub. Date: Oct. 13, 2005

(54) Bazedoxifene acetate formulations

(75) Inventors: Syed M. Shah, East Hanover, NJ (US); Kadum A. Ali, Congers, NY (US); Christian L. Ofslager, Newburgh, NY (US); Mahdi B. Fawzi, Morristown, NJ (US)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/100,693

(22) Filed: Apr. 7, 2005

Related U.S. Application Data
(60) Provisional application No. 60/560,452, filed on Apr. 8, 2004.

(51) Int. Cl. A61K 31/55

(52) U.S. Cl. 514/217.09

(57) ABSTRACT

The present invention is directed to solid dispersions of bazedoxifene acetate, compositions containing the same, preparations thereof, and uses thereof.

### CROSS REFERENCE TO RELATED APPLICATIONS

This application claims the benefit of U.S. Ser. No. 60/560,452, filed Apr. 8, 2004, the disclosure of which is incorporated herein by reference in its entirety.

### FIELD OF THE INVENTION

The present invention relates to solid dispersions and compositions thereof of the selective estrogen receptor modulator 1 -[4-(2-azepan-1-yl-ethoxy)-benzyl]-2-(4-hydroxy-phenyl)-3-methyl-1H-indol-5-ol acetic acid (bazedoxifene acetate).

### BACKGROUND OF THE INVENTION

Bazedoxifene acetate (1-[4-(2-azepan-1-yl-ethoxy)-benzyl]-2-(4-hydroxy-phenyl)-3-methyl-1H-indol-5-ol acetic acid), having the chemical formula shown below: 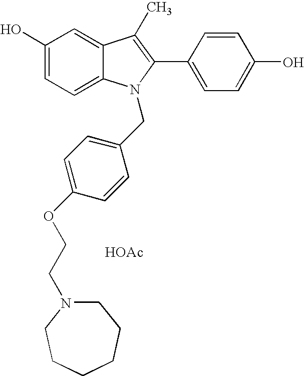
belongs to the class of drugs typically referred to as selective estrogen receptor modulators (SERMs). Consistent with its classification, bazedoxifene demonstrates affinity for estrogen receptors (ER) but shows tissue selective estrogenic effects. For example, bazedoxifene acetate demonstrates little or no stimulation of uterine response in preclinical models of uterine stimulation. Conversely, bazedoxifene acetate demonstrates an estrogen agonist-like effect in preventing bone loss and reducing cholesterol in an ovariectomized rat model of osteopenia. In an MCF-7 cell line (human breast cancer cell line), bazedoxifene acetate behaves as an estrogen antagonist. These data demonstrate that bazedoxifene acetate is estrogenic on bone and cardiovascular lipid parameters and antiestrogenic on uterine and mammary tissue and thus has the potential for treating a number of different disease or disease-like states wherein the estrogen receptor is involved.

U.S. Pat. Nos. 5,998,402 and 6,479,535 report the preparation of bazedoxifene acetate. The synthetic preparation of bazedoxifene acetate has also appeared in the general literature. See, for example, Miller et al., J. Med. Chem., 2001, 44, 1654-1657. Further description of the drug's biological activity has appeared in the general literature as well (e.g. Miller, et al. Drugs of the Future, 2002, 27(2), 117-121). Formulations of bazedoxifene acetate are also reported in U.S. Pat. App. Pub. No. 2002/0031548 A1.

Because drug formulations showing, for example, improved bioavailability are consistently sought, there is an ongoing need for new formulations of existing drug molecules. The solid dispersions of bazedoxifene acetate and compositions containing the same described herein helps meet these and other needs.

### SUMMARY OF THE INVENTION

In some embodiments, the present invention provides a solid dispersion comprising bazedoxifene acetate dispersed in a dispersing agent.

In some embodiments, the present invention provides a composition comprising the solid dispersion described herein and a pharmaceutically acceptable carrier.

In some embodiments, the present invention provides a dosage form comprising the solid dispersion described herein.

In some embodiments, the present invention provides a method of preparing the solid dispersion described herein, comprising: a) combining bazedoxifene acetate and a dispersing agent in solution; and b) removing solvent to yield the solid dispersion.

In some embodiments, the present invention provides a method of preparing the solid dispersion described herein, comprising: a) combining bazedoxifene acetate with melted dispersing agent to form a liquid mixture; and b) solidifying the liquid mixture to form the solid dispersion.

In some embodiments, the present invention provides a method of treating a mammal having a disease or syndrome associated with estrogen deficiency or excess of estrogen comprising administering to the mammal a therapeutically effective amount of the solid dispersion of described herein.

In some embodiments, the present invention provides a method of treating a mammal having a disease or disorder associated with proliferation or abnormal development of endometrial tissues comprising administering to the mammal a therapeutically effective amount of the solid dispersion described herein.

In some embodiments, the present invention provides a method of lowering cholesterol in a mammal comprising administering to the mammal a therapeutically effective amount of the solid dispersion described herein.

In some embodiments, the present invention provides a method of inhibiting bone loss in a mammal comprising administering to the mammal a therapeutically effective amount of the solid dispersion described herein.

In some embodiments, the present invention provides a method of treating breast cancer in a mammal comprising administering to the mammal a therapeutically effective amount of the solid dispersion described herein.

In some embodiments, the present invention provides a method of treating postmenopausal woman for one or more vasomotor disturbances comprising administering to the postmenopausal woman a therapeutically effective amount of the solid dispersion of described herein.

### BRIEF DESCRIPTION OF THE DRAWINGS

FIG. 1 shows a plot comparing dissolution rates of bazedoxifene acetate as a crystalline solid and as a solid dispersion with PVP according to Example 3.

FIG. 2 shows a plot comparing bioavailability of bazedoxifene acetate in dogs for formulations containing dispersion and non-dispersion formulations according to Example 4.

### DETAILED DESCRIPTION

The present invention provides, inter alia, bazedoxifene acetate (BZA) solid dispersions and compositions thereof having improved properties relating to solubility, bioavailability and the like. The solid dispersions of the invention have increased solubility and bioavailability compared with, for example, crystalline BZA or microcrystalline BZA. The increased bioavailability associated with solid BZA dispersions has numerous advantages including allowing for administration of lower dosages, thereby lessening chances for adverse side effects and reducing subject variability.

The compositions of the invention contain, for example, BZA dispersed in a dispersing agent. In some embodiments, the weight ratio of BZA to dispersing agent is about 1:99 to about 99:1. In some embodiments, the weight ratio of BZA to dispersing agent is about 1:99 to about 75:25 or about 1:99 to about 60:40. In further embodiments, the weight ratio of BZA to dispersing agent is about 1:99 to about 15:85; about 1:99 to about 10:90; or about 1:99 to about 5:95. In further embodiments, the weight ratio of BZA to dispersing agent is about 5:95. In further embodiments, the weight ratio of BZA to dispersing agent is about 25:75 to about 75:25, about 40:60 to about 60:40 or about 1:1. In some embodiments, the weight ratio of BZA to dispersing agent is about 1:1.

The “dispersing agent,” as used herein, refers to any substance or mixture of substances that acts as a dispersing medium for molecules/particles of bazedoxifene acetate. The dispersing agent is typically composed of a pharmaceutically acceptable substance that does not substantially interfere with the pharmaceutical action of BZA. The phrase “pharmaceutically acceptable” is employed herein to refer to those substances which are, within the scope of sound medical judgment, suitable for use in contact with the tissues of human beings and animals without excessive toxicity, irritation, allergic response, or other problem or complication, commensurate with a reasonable benefit/risk ratio. In some embodiments, the dispersing agent is a solid at room temperature (e.g., about 22° C.). In further embodiments, the dispersing agent melts at a temperature between about 30 and 100° C. In further embodiments, the dispersing agent is soluble in an organic solvent.

Non-limiting examples of suitable dispersing agents include polymers such as celluloses (e.g., carboxymethylcelluloses, methylcelluloses, hydroxypropylcelluloses. hydroxypropylmethylcelluloses); hyaluronates; alginates; polysaccharides, heteropolysaccharides (pectins); poloxamers; poloxamines; ethylene vinyl acetates; polyethylene glycols; dextrans; polyvinylpyrrolidones; chitosans; polyvinylalcohols; propylene glycols; polyvinylacetates; phosphatidylcholines (lecithins); miglyols; polylactic acid; polyhydroxybutyric acid; mixtures of two or more thereof, copolymers thereof, derivatives thereof, and the like. Further example dispersing agents include copolymer systems such as polyethylene glycol-polylactic acid (PEG-PLA), polyethylene glycol-polyhydroxybutyric acid (PEG-PHB), polyvinylpyrrolidone-polyvinylalcohol (PVP-PVA), and derivatized copolymers such as copolymers of N-vinyl purine (or pyrimidine) derivatives and N-vinylpyrrolidone.

In some embodiments, the dispersing agent contains polyvinylpyrrolidone (PVP) or derivative thereof. PVP is a polyamide that forms complexes with a wide variety of substances and is considered to be chemically and physiologically inert. Examples of suitable PVPs include polyvinylpyrrolidones having an average molecular weight from about 10,000 to about 50,000. In some embodiments, the polyvinylpyrrolidone has an average molecular weight of about 10,000 to about 20,000. In further embodiments, the polyvinylpyrrolidone has a molecular weight of about 15,000 to about 20,000. An example suitable PVP is PVP K-17 (PLASDONE povidone, ISP Technologies, Ltd.). In some embodiments, the dispersing agent consists essentially of PVP or derivative thereof.

In some embodiments, the dispersing agent contains a block co-polymer of ethylene and propylene glycol, often referred to as a Poloxamer. Some suitable example Poloxamers include Poloxamer 188 (LUTROL F 68, BASF), Poloxamer 407 (LUTROL F 127, BASF). and the like. In some embodiments, the dispersing agent is Poloxamer 188.

In some embodiments, the dispersing agent contains a polyethylene glycol (PEG). Suitable PEGs include PEG 200, 300, 400, 600, 1000, 1450, 3350, 4000, 6000, 8000, 10000, 20000, mixtures thereof and the like. In some embodiments, the dispersing agent is PEG 1450.

The BZA dispersions of the invention can be made by any of numerous methods that result in, for example, a solid dispersion of amorphous BZA. In an example method, BZA (in any form, e.g., crystalline, amorphous, etc.) and the dispersing agent can be dissolved in a dispersing solvent (together, or separately and then combined) in the weight ratio desired and then the dispersing solvent is removed to yield the desired solid dispersion. The dispersing solvent can be an aqueous solvent or organic solvent. Suitable organic solvents include alcohols, ethers, hydrocarbons, halogenated hydrocarbons, nitrites, mixtures thereof, and the like. In some embodiments, the organic solvent is a volatile solvent such as methanol, ethanol, isopropanol, diethyl ether, pentane, hexane, benzene, dichloromethane, acetonitrile, mixtures thereof and the like. In some embodiments, the organic solvent is an alcohol such as methanol, ethanol, n-propanol, ispropanol, mixtures thereof and the like. In some embodiments, the organic solvent is ethanol.

In another example, BZA and dispersing agent can be combined in the desired weight ratio when either or both the BZA and dispersing agent is (are) in liquid form (e.g., a melt), and then the liquid mixture is solidified to form the desired solid dispersion. According to such embodiments, the BZA and dispersing agent can be combined when at least one of the BZA and dispersing agent is melted. The resulting mixture is then solidified by cooling to a temperature sufficient to solidify the mixture. In some embodiments, the mixture is cooled to about 25° C. or below. In some embodiments, BZA is combined with melted dispersing agent and the resulting mixture cooled to a temperature below the melting point of the mixture to form the solid dispersion. In further embodiments, the dispersing agent is heated to a temperature between about 30 and 200° C., between about 30 and 150° C., or between about 30 and 100° C., which is a temperature that is at or above the melting point of the dispersing agent. In further embodiments, the dispersing agent is heated to a temperature above about 30, above about 40, above about 50, above about 60, above about 70, above about 80 or above about 90° C. These and other methods are routine techniques suitable for the preparation of the BZA dispersions of the invention.

In some embodiments, the solid dispersions of the invention are characterized by an equilibrium solubility in 0.0005 M acetic acid at a temperature of about 20 to about 26° C. that is greater than that for crystalline or microcrystalline bazedoxifene acetate. In further embodiments, the solid dispersions of the invention are characterized by an equilibrium solubility in 0.0005 M acetic acid at a temperature of about 20 to about 26° C. that is at least about 8, at least about 10, at least about 12, at least about 14, at least about 16, or at least about 19 mg/mL. Equilibrium solubility can be measured by routine methods in the art such as described in Example 2.

In some embodiments, the solid dispersions of the invention are characterized such that a dosage form comprising about 10 mg total of bazedoxifene acetate in a solid dispersion is characterized by an AUC₀₋₂₄ greater than about 140, greater than about 150, greater than about 160, greater than about 170, or greater than about 180 ng·hr/mL when orally administered to mammal. In further embodiments, the solid dispersions of the invention are characterized such that a dosage form comprising about 10 mg total of bazedoxifene acetate in a solid dispersion is characterized by:

a) an AUC₀₋₂₄ of about 140 to about 250 ng·hr/mL;

b) a Cₘₐₓ of about 12 to about 30 ng/mL; and

c) a tₘₐₓ of about 1.0 to about 3.5 hr;

when orally administered to mammal. Methods for measuring the pharmacokinetic parameters AUC₀₋₂₄ (area under curve for 24 hours), Cₘₐₓ, and tₘₐₓ are well known in the art and described, for example, in Example 4.

Dosage and Formulation

The solid dispersions described herein can be formulated for administration to a patient in any of a variety of ways. In some embodiments, the solid dispersions can be administered alone, i.e., without the addition of excipients or other additives. For example, solid dosage forms (e.g., tablet, capsules etc.) containing greater than about 95%, greater than about 98%, or greater than about 99% (by weight) of solid dispersion described herein can be directly administered to a patient.

In some embodiments, the solid dispersions are combined with one or more pharmaceutically acceptable carriers (excipients) to form a pharmaceutical composition for administration to a patient. The composition can contain any amount of solid dispersion. In some embodiments, the compositions contains about 1 to about 99% by weight of the solid dispersion. In further embodiments, the composition contains about 1 to about 50% by weight of the solid dispersion. In yet further embodiments, the composition contains about 1 to about 30% by weight of the solid dispersion. In yet further embodiments, the composition contains about 1 to about 20% by weight of the solid dispersion. In yet further embodiments, the composition contains about 1 to about 10% by weight of the solid dispersion.

Formulations containing the present solid dispersions can be administered in daily doses ranging from 0.1 mg to 1000 mg of bazedoxifene acetate to a person in need. Preferred dose ranges vary from 10 mg/day to about 600 mg/day, more preferably from 10 mg/day to about 60 mg/day. The dosing can be either in a single dose or two or more divided doses per day. Such doses can be administered in any manner that facilitates the compound's entry into the bloodstream including orally, via implants, parenterally, vaginally, rectally, and transdermally.

Transdermal administrations include all administrations across the surface of the body and the inner linings of body passages including epithelial and mucosal tissues. Such administration may be in the form of a lotion, cream, colloid, foam, patch, suspension, and the like.

Oral formulations containing the present solid dispersions can comprise any conventionally used oral forms, including tablets, capsules, buccal forms, troches, lozenges and oral liquids, suspensions, and the like. Capsules or tablets of containing the present solid dispersion can also be combined with mixtures of other active compounds or inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc.

Tablet formulations can be made by conventional compression, wet granulation, or dry granulation methods and utilize pharmaceutically acceptable diluents (fillers), binding agents, lubricants, disintegrants, suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Oral formulations used herein may utilize standard delay or time release formulations or spansules. Suppository formulations may be made from traditional materials, including cocoa butter, with or without the addition of waxes to alter the suppositories melting point, and glycerin. Water soluble suppository bases, such as polyethylene glycols of various molecular weights, may also be used.

Film coatings useful with the present formulations are known in the art and generally consist of a polymer (usually a cellulosic type of polymer), a colorant and a plasticizer. Additional ingredients such as wetting agents, sugars, flavors, oils and lubricants can be included in film coating formulations to impart certain characteristics to the film coat. The compositions and formulations herein may also be combined and processed as a solid, then placed in a capsule form, such as a gelatin capsule.

The filler or diluent can comprise any substance known in the art that is useful for the preparation of solid oral formulations. Pharmaceutically acceptable fillers can be selected from, for example, lactose, microcrystalline cellulose, sucrose, mannitol, calcium phosphate, calcium carbonate, powdered cellulose, maltodextrin, sorbitol, starch, xylitol, and the like.

The present formulations can also include disintegrant agents. These disintegrants can be selected from those known in the art, including pregelatinized starch, sodium starch glycolate and the like. Other useful disintegrants include croscarmellose sodium, crospovidone, starch, alginic acid, sodium alginate, clays (e.g. veegum or xanthan gum), cellulose floc, ion exchange resins, or effervescent systems, such as those utilizing food acids (such as citric acid, tartaric acid, malic acid, fumaric acid, lactic acid, adipic acid, ascorbic acid, aspartic acid, erythorbic acid, glutamic acid, and succinic acid) and an alkaline carbonate component (such as sodium bicarbonate, calcium carbonate, magnesium carbonate, potassium carbonate, ammonium carbonate, etc.). The disintegrant(s) useful herein can comprise from about 4% to about 40% of the composition by weight, preferably from about 15% to about 35%, more preferably from about 20% to about 35%.

Some components can have multiple functions in the formulations of this invention, acting e.g. as both a filler and a disintegrant, and its function in a specific formulation may be singular even though its properties may allow multiple functionality.

The pharmaceutical formulations and excipient systems herein can also contain an antioxidant or a mixture of antioxidants, such as ascorbic acid. Other antioxidants which can be used include sodium ascorbate and ascorbyl palmitate, optionally in conjunction with an amount of ascorbic acid. An example range for the antioxidant(s) is from about 0.05% to about 15% by weight, from about 0.5% to about 15% by weight, or from about 0.5% to about 5% by weight. In some embodiments, the pharmaceutical formulations contain substantially no antioxidant.

Pharmaceutical compositions of the present solid dispersions can also be formulated with steroidal estrogens, such as conjugated estrogens, USP. The amount of bazedoxifene acetate used in the formulation can be adjusted according to the particular solid dispersion used, the amount and type of steroidal estrogen in the formulation as well as the particular therapeutic indication being considered. In general, the bazedoxifene acetate can be used in an amount sufficient to antagonize the effect of the particular estrogen to the level desired. The dose range of conjugated estrogens can be from about 0.3 mg to about 2.5 mg, about 0.3 mg to about 1.25 mg, or about 0.3 mg to about 0.625 mg. An example range for amount of bazedoxifene acetate in a combination formulation is about 10 mg to about 40 mg. For the steroidal estrogen mestranol, a daily dosage can be from about 1 μG to about 150 μG, and for ethynyl estradiol a daily dosage of from about 1 μG to 300 μG can be used. In some embodiments, the daily dose is between about 2 μG and about 150 μG.

An example oral formulation contains the present solid dispersion and the following excipient systems:

a) a filler and disintegrant together comprising from about 1% to about 99% by weight (wt) of the total formulation, preferably between about 20% and about 85% of the formulation, of which from about 4% to about 45% by weight of the total formulation; and

b) a lubricant comprising from about 0.2% to about 15% of the composition (wt), where the lubricant is magnesium stearate or other metallic stearates (e.g. calcium stearate or zinc stearate), fatty acid esters (e.g. sodium stearyl fumarate), fatty acids (e.g. stearic acid), fatty alcohols, glyceryl behenate, mineral oil, parrafins, hydrogenated vegetable oils, leucine, polyethylene glycols, metallic lauryl sulfates or sodium chloride.

The percentages listed above for the filler, disintegrant, and lubricants are based on final pharmaceutical composition. The remainder of the final composition is comprised of the solid dispersion and a pharmaceutically acceptable surface covering, such as a coating or capsule, as described herein. In some embodiments of this invention, the solid dispersion comprises from about 1% to about 99%, about 10 to about 95%, or about 20 to about 90% by weight, of the final composition; and the coating or capsule comprises up to about 8%, by weight, of the formulation.

Additional numerous various excipients, dosage forms, dispersing agents and the like that are suitable for use in connection with the solid dispersions of the invention are known in the art and described in, for example, Remington's Pharmaceutical Sciences, 17th ed., Mack Publishing Company, Easton, Pa., 1985, which is incorporated herein by reference in its entirety.

Methods

As described in U.S. Pat. No. 5,998,402, bazedoxifene and salts thereof are selective estrogen agonists with affinity for the estrogen receptor. Unlike other types of estrogen agonists, bazedoxifene and salts thereof are antiestrogenic in the uterus and can antagonize the trophic effects of estrogen agonists in uterine tissues. Accordingly, the solid dispersions of the invention, and compositions containing the same, can find many uses related to treating disease states or syndromes associated with an estrogen deficiency or an excess of estrogen. They may also be used in methods of treatment for diseases or disorders which result from proliferation or abnormal development, actions or growth of endometrial or endometrial-like tissues.

Bazedoxifene acetate has the ability to behave like an estrogen agonist by lowering cholesterol and preventing bone loss. Accordingly, the solid dispersion is useful for treating many maladies which result from estrogen effects and estrogen excess or deficiency including osteoporosis, prostatic hypertrophy, male pattern baldness, vaginal and skin atrophy, acne, dysfunctional uterine bleeding, endometrial polyps, benign breast disease, uterine leiomyomas, adenomyosis, ovarian cancer, infertility, breast cancer, endometriosis, endometrial cancer, polycystic ovary syndrome, cardiovascular disease, contraception, Alzheimer's disease, cognitive decline and other CNS disorders, as well as certain cancers including melanoma, prostrate cancer, cancers of the colon, CNS cancers, among others. Additionally, the solid dispersion can be used for contraception in pre-menopausal women, as well as hormone replacement therapy in post-menopausal women (such as for treating vasomotor disturbances such as hot flush) or in other estrogen deficiency states where estrogen supplementation would be beneficial. It can also be used in disease states where amenorrhea is advantageous, such as leukemia, endometrial ablations, chronic renal or hepatic disease or coagulation diseases or disorders.

The solid dispersions of the invention can also be used in methods of inhibition of bone loss, which can result from an imbalance in a individual's formation of new bone tissues and the resorption of older tissues, leading to a net loss of bone. Such bone depletion results in a range of individuals, particularly in post-menopausal women, women who have undergone bilateral oophorectomy, those receiving or who have received extended corticosteroid therapies, those experiencing gonadal dysgenesis, and those suffering from Cushing's syndrome. Special needs for bone, including teeth and oral bone, replacement can also be addressed using the present solid dispersion in individuals with bone fractures, defective bone structures, and those receiving bone-related surgeries and/or the implantation of prosthesis. In addition to the problems described above, the solid dispersion can be used in treatments for osteoarthritis, hypocalcemia, hypercalcemia, Paget's disease, osteomalacia, osteohalisteresis, multiple myeloma and other forms of cancer having deleterious effects on bone tissues.

Methods of treating the diseases and syndromes listed herein are understood to involve administering to an individual in need of such treatment a therapeutically effective amount of the solid dispersion of the invention, or composition containing the same. As used herein, the term “treating” in reference to a disease is meant to refer to preventing, inhibiting and/or ameliorating the disease.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) preventing the disease; for example, preventing a disease, condition or disorder in an individual that may be predisposed to the disease, condition or disorder but does not yet experience or display the pathology or symptomatology of the disease;

(2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and

(3) ameliorating the disease; for example, ameliorating a disease, condition or disorder in an individual that is experiencing or displaying the pathology or symptomatology of the disease, condition or disorder (i.e., reversing the pathology and/or symptomatology).

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

### Preparation of Bazedoxifene Acetate Solid Dispersions

Solid dispersion formulations of bazedoxifene acetate were prepared according to the procedures set forth below. The dispersions were all found to be amorphous (non crystalline) by X-ray powder diffraction, which is in contrast to a physical mixture of BZA and dispersing reagent which was shown to contain crystalline BZA.

### Example 1.1

### Bazedoxifene Acetate Solid Dispersion with PVP (1:1 w/w)

To a solution of 3.0004 g of PVP K17 (PLASDONE, Povidone USP Polyvinylpyrrolidone, ISP Technologies Inc.) in 55 mL of ethanol (EM Science) was added 3.0891 g of bazedoxifene acetate. Another 20 mL of ethanol was added and the resulting suspension was warmed to 65° C. for 5 minutes until a clear brown solution was observed. Solvents were evaporated under reduced pressure at room temperature to dryness. The yellow flakes were collected and grinded with mortar and pestle to give 5.6 g of brown-creamy powder.

### Example 1.2

### Bazedoxifene Acetate Solid Dispersion with PVP (1:1 w/w)

To a solution of 2.1091 g of PVP K17 (PLASDONE, Povidone USP. Polyvinylpyrrolidone, ISP Technologies Inc.) in 4 mL of ethanol was added 2.1028 g of bazedoxifene acetate. Another 2 mL of ethanol was added to form a milky suspension. Another 44 mL of ethanol was added (total of 50 mL) and the mixture was heated to 65° C. for 5 min to make a yellow solution. Solvents were evaporated under reduced pressure at room temperature to dryness to give a yellow-brown solid.

### Example 1.3

### Bazedoxifene Acetate Solid Dispersion with PVP (1:1 w/w)

To a solution of 3.00519 g of PVP K17 in 15 mL of ethanol was added 3.00671 g of bazedoxifene acetate with mixing. Another 60 mL of ethanol was added and the mixture was warmed to 65° C. for 5 minutes to get a clear yellow-brown solution. Solvents were evaporated under reduced pressure at room temperature to dryness. The yellow-brown solid was grinded with mortar and pestle to give yellow-creamy fine powder.

### Example 1.4

### Bazedoxifene Acetate Solid Dispersion with PVP (5% w/w Active)

To a solution of 0.9509 g of PVP K-17 (PLASDONE, Povidone USP, Polyvinylpyrrolidone, ISP Technologies Inc.) in 1 mL of ethanol (EM Science) was added 0.0499 g of bazedoxifene acetate. A thick yellow solution was formed and 0.5 mL of ethanol was added to the mixture to form a yellow viscous solution. Solvents were evaporated under reduced pressure at room temperature to dryness. The yellow solid material was collected and grinded with mortar and pestle.

### Example 1.5

### Bazedoxifene Acetate Solid Dispersion with Poloxamer 188 (5% w/w Active)

To a solution of 0.9503 g of Poloxamer 188 (BASF; polyoxypropylene-polyoxyethylene copolymer) in 1.5 mL of ethanol and 0.5 mL of de-ionized water, was added 0.0503 g of bazedoxifene acetate to form a colorless solution. Solvents were evaporated under reduced pressure at room temperature to dryness. The creamy solid material was collected.

### Example 1.6

### Bazedoxifene Acetate Solid Dispersion with Poloxamer 188 (5% w/w Active)

To Poloxamer 188 (0.9540 g; BASF) melted at 60° C. was added 0.0502 g of bazedoxifene acetate with mixing to form a clear liquid. The liquid was cooled to room temperature.

### Example 1.7

### Bazedoxifene Acetate Solid Dispersion with PEG 1450 (5% w/w Active)

To a solution of 0.9522 g of PEG 1450 (Union Carbide) in 1.5 mL of ethanol heated to 40° C. was added 0.0510 g of bazedoxifene acetate to form a clear solution. Solvents were evaporated under reduced pressure to dryness to form white waxy material.

### Example 1.8

### Bazedoxifene Acetate Solid Dispersion with PEG 1450 (5% w/w Active)

To 0.9448 g of melted PEG 1450 at 70° C. was added 0.0504 g of bazedoxifene acetate with mixing to form a clear liquid. The liquid was cooled to room temperature.

### Example 2

### Equilibrium Solubility of Bazedoxifene Acetate Solid Dispersions

A few milligrams of each of the dispersions of Examples 1.1 to 1.8 were placed in 2 mL of 0.0005 M acetic acid, held at room temperature (about 20-26° C.), rotated at 50 rotation/min for 18 hours, and filtered through 0.45 μm (Nylon Acrodisc) filter. After 10× dilution with mobile phase, 10 μL was injected to HPLC. The HPLC was carried out with the following parameters:
Column: Inertsil 5 ODS-2 150×4.6 mm
Flow rate: 1.5 mL/min
Detector: UV at 220 nm
Temperature: Ambient
Mobile Phase: 320 mL of acetonitrile and 680 mL of a solution containing 6.8 g of monobasic potassium phosphate in 2 L water, pH adjusted to 3.0 with 85% phosphoric acid).

Results are provided in Table II below. Bazedoxifene solid dispersion with PVP (5% w/w active), gave the highest equilibrium solubility.

TABLE II

| Dispersion Example No. | Equilibrium Solubility |
|---|---|
| 1.1 (ethanol; PVP; 1:1 w/w) | 13.9 mg/mL |
| 1.4 (ethanol; PVP; 5% w/w active) | 20.1 mg/mL |
| 1.5 (ethanol; Poloxamer 188; 5% w/w active) | 2.9 mg/mL |
| 1.6 (melt; Poloxamer 188; 5% w/w active) | 8.1 mg/mL |
| 1.7 (ethanol; PEG 1450; 5% w/w active) | 2.3 mg/mL |
| 1.8 (melt; PEG 1450; 5% w/w active) | 5.3 mg/mL |

### Example 3

### Intrinsic Dissolution Rate of Bazedoxifene Acetate versus Bazedoxifene Acetate Solid Dispersion with PVP (1:1 w/w)

Pellets of each of bazedoxifene acetate and bazedoxifene acetate solid dispersion (PVP; 1:1 w/w, see Example 1) were prepared by compressing 100 mg of each in a die (Wood's Apparatus) at 1000 psi pressure for 1 minute with a Carver press. The pellets were then fitted into a dissolution apparatus which resulted in a single exposed surface of pellet with a surface area of 0.5 cm². The dissolution rate in 900 mL of 0.0005 M acetic acid was determined using the USP method (apparatus 2) with a rotation of 50 rpm at 37° C. From the concentration of mg/mL (by HPLC) versus time profile, the apparent intrinsic dissolution rate was determined.

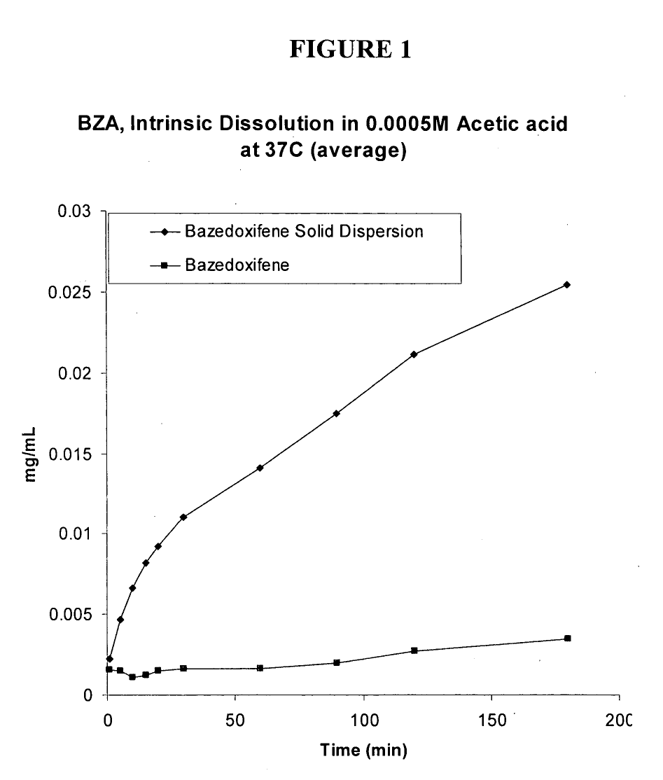

The intrinsic dissolution rates of bazedoxifene acetate and bazedoxifene acetate solid dispersion with PVP (1:1 w/w) were 0.018 mg/cm²-min and 0.18 mg/cm²-min, respectively. The bazedoxifene acetate solid dispersion is about 10 times faster than the non-dispersed material. Results are shown in FIG. 1.

### Example 4

### Preliminary Pharmacokinetic Analysis of Bazedoxifene Acetate Solid Dispersions in Dogs

Bioavailability of bazedoxifene acetate formulations was evaluated in dogs. Six female dogs (6.2-10.5 kg) were divided into three groups, 2 dogs per group. To each group of dogs, a single dose equivalent to 10 mg of bazedoxifene acetate was administered orally as one of three formulations:

Formulation A) ½ of one 20 mg tablet (Table III);

Formulation B) one 10 mg capsule of bazedoxifene acetate solid dispersion according to Ex. 1.1 where the capsule has the same formula as for formulation A but without SLS (Table IV); and

Formulation C) one 10 mg capsule containing bazedoxifene acetate solid dispersion according to Ex. 1.1 (i.e., without excipients) (Table V).

The study was conducted as a randomized, crossover study. The formulations were administered following an overnight fast, and food was offered following the four hour blood sample. Blood samples were drawn at 0 (predose), 0.25, 0.5, 1, 1.5, 2, 3, 4, 6, 8, 12 and 24 hours after dosing; and plasma was separated and assayed for bazedoxifene acetate content.

The compositions of batches A, B and C are provided below in Tables III, IV, and V, respectively. Capsules were prepared by mixing components in bag blends and filling the capsule manually (a Capsogel #2 CS, white opaque capsule).

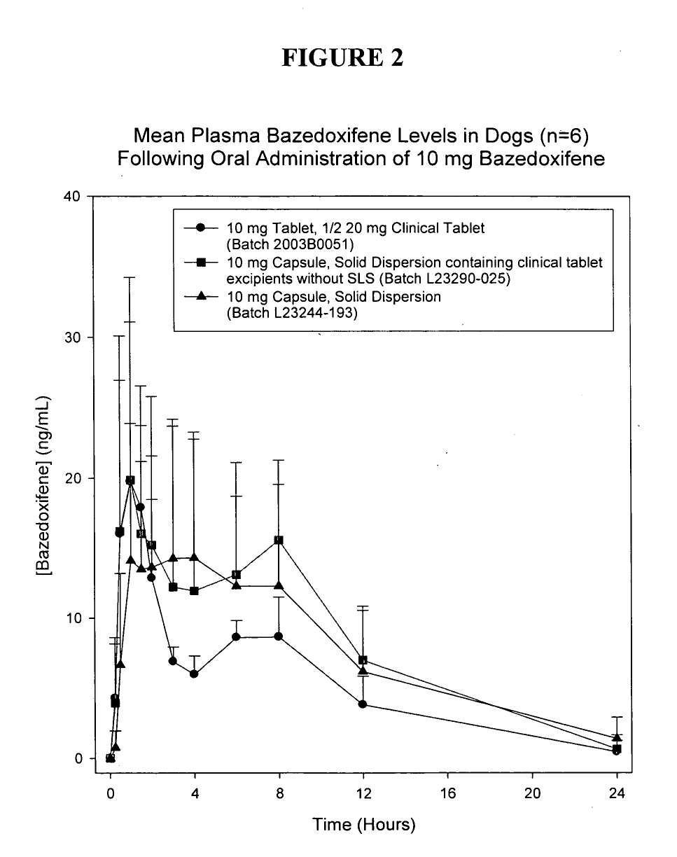

Results are provided in Table VI and FIG. 2. As can be seen from the AUC (area under curve) data, bioavailability of bazedoxifene acetate when formulated as a dispersion was found to be about 50% higher than for non-dispersion formulations.

TABLE III
Formulation A

| Ingredient | % w/w | mg/tablet |
|---|---|---|
| bazedoxifene acetate, micronized | 4.843 | 20.00 |
| lactose, NF | 35.206 | 145.40 |
| microcrystalline cellulose (Avicel PH 101) | 33.898 | 140.00 |
| pregelatinized starch, NF (Starch 1500) | 13.559 | 56.00 |
| sodium lauryl sulfate (SLS) | 1.453 | 6.00 |
| sodium starch glycolate, NE | 5.811 | 24.00 |
| ascorbic acid | 1.453 | 6.00 |
| silicon dioxide (Syloid 244 FP) | 0.145 | 0.60 |
| magnesium stearate, NF | 0.484 | 2.00 |
| White Opadry 1 | 3.148 | 13.00 |
| Total | 100.00 | 413.00 |

TABLE IV
Formulation B

| | | | g/15 |
|---|---|---|---|
| Ingredient | % w/w | mg/capsule | gram batch |
| bazedoxifene acetate solid | 11.23 | 22.13 | 1.6850 |
| dispersion of Example 1.1 | | | |
| (45.191 % use at value) | | | |
| lactose, NF | 31.00 | 61.07 | 4.6500 |
| microcrystalline cellulose | 35.53 | 70.00 | 5.3299 |
| (Avicel PH 101) | | | |
| pregelatinized starch, NF | 14.21 | 28.00 | 2.1320 |
| (Starch 1500) | | | |
| sodium starch glycolate, NF | 6.09 | 12.00 | 0.9137 |
| ascorbic acid | 1.52 | 3.00 | 0.2284 |
| silicon dioxide (Syloid 244 FP) | 0.15 | 0.30 | 0.0228 |
| magnesium stearate, NE | 0.25 | 0.50 | 0.0381 |
| Total | 100.00 | 197.00 | 15.0000 |

TABLE V
Formulation C

| Ingredient | % w/w | mg/capsule |
|---|---|---|
| bazedoxifene acetate solid | 100 | 22.13 |
| dispersion of Example 1.1 | | |
| (45.191% use at value) | | |

TABLE VI
Mean (% CV) Bazedoxifene Acetate Pharmacokinetic
Parameters in Dogs Following Single Dose Oral
Administration Equivalent to 10 mg Bazedoxifene Acetate

| | | Formulation B | Formulation C |
|---|---|---|---|
| | | Solid Dispersion | Solid Dispersion |
| | Formulation A | Capsule without | Capsule without |
| Parameter | Tablet | SLS | excipients |
| AUC₀₋₂₄ | 124 (19) | 188 (52) | 173 (56) |
| (ng · hr/mL) | | | |
| Cₘₐₓ (ng/mL) | 21.8 (43) | 26.0 (37) | 16.9 (57) |
| tₘₐₓ (hr) | 2.33 (120) | 1.42 (65) | 2.42 (59) |

Various modifications of the invention, in addition to those described herein, will be apparent to those skilled in the art from the foregoing description. Such modifications are also intended to fall within the scope of the appended claims. Each of the publications and, references, including books and patents, cited in the present application is incorporated herein by reference in its entirety.

1. A solid dispersion comprising bazedoxifene acetate dispersed in a dispersing agent.

2. The solid dispersion of claim 1 wherein said bazedoxifene acetate in said solid dispersion is amorphous.

3. The solid dispersion of claim 1 wherein said dispersing agent comprises a cellulose, hyaluronate, alginate, polysaccharide, heteropolysaccharide, poloxamers, poloxamines, ethylene vinyl acetate, polyethylene glycol, dextran, polyvinylpyrrolidone, chitosan, polyvinylalcohol, propylene glycol, polyvinylacetate, phosphatidylcholines, miglyol, polylactic acid, polyhydroxybutyric acid, mixtures of two or more thereof or copolymers thereof.

4. The solid dispersion of claim 3 wherein said dispersing agent comprises polyvinylpyrrolidone, poloxamer or polyethylene glycol.

5. The solid dispersion of claim 4 wherein said dispersing agent comprises polyvinylpyrrolidone.

6. The solid dispersion of claim 4 wherein said dispersing agent comprises Poloxamer 188.

7. The solid dispersion of claim 4 wherein said dispersing agent comprises PEG 1450.

8. The solid dispersion of claim 1 wherein the weight ratio of bazedoxifene acetate to dispersing agent is about 1:99 to about 75:25.

9. The solid dispersion of claim 1 wherein the weight ratio of bazedoxifene acetate to dispersing agent is about 1:99 to about 60:40.

10. The solid dispersion of claim 1 wherein the weight ratio of bazedoxifene acetate to dispersing agent is about 1:99 to about 10:90.

11. The solid dispersion of claim 1 wherein the weight ratio of bazedoxifene acetate to dispersing agent is about 5:95.

12. The solid dispersion of claim 1 wherein the weight ratio of bazedoxifene acetate to dispersing agent is about 40:60 to about 60:40.

13. The solid dispersion of claim 1 wherein the weight ratio of bazedoxifene acetate to dispersing agent is about 1:1.

14. The solid dispersion of claim 1 having an equilibrium solubility in 0.0005 M acetic acid at a temperature of about 20 to about 26° C. of at least about 8 mg/mL.

15. The solid dispersion of claim 1 wherein a dosage form comprising about 10 mg total of bazedoxifene acetate in the form of said solid dispersion is characterized by an AUC₀₋₂₄ greater than about 140 ng·hr/mL when orally administered to mammal.

16. The solid dispersion of claim 1 wherein a dosage form comprising about 10 mg total of bazedoxifene acetate in the form of said solid dispersion is characterized by:
a) an AUC₀₋₂₄ of about 140 to about 250 ng·hr/mL;
b) a Cₘₐₓ of about 12 to about 30 ng/mL; and
c) a tₘₐₓ of about 1.0 to about 3.5 hr;
when orally administered to mammal.

17. A method of preparing the solid dispersion of claim 1 comprising:
a) combining bazedoxifene acetate and said dispersing agent in solution, wherein said solution comprises a solvent; and
b) removing said solvent to yield said solid dispersion.

18. The method of claim 17 wherein said solvent is an organic solvent.

19. The method of claim 18 wherein said organic solvent comprises an alcohol.

20. The method of claim 19 wherein said alcohol comprises ethanol.

21. A solid dispersion prepared by the method of claim 17.

22. A method of preparing the solid dispersion of claim 1 comprising:
a) combining bazedoxifene acetate with melted dispersing agent to form a liquid mixture; and
b) solidifying said liquid mixture to form said solid dispersion.

23. The method of claim 22 wherein said melted dispersing agent is prepared by heating said dispersing agent to a temperature above about 30° C.

24. The method of claim 22 wherein said solidifying is carried out by cooling said liquid mixture to a temperature at or below about 25° C.

25. A solid dispersion prepared by the method of claim 22.

26. A composition comprising the solid dispersion of claim 1 and a pharmaceutically acceptable carrier.

27. The composition of claim 26 comprising about 1 to about 99% by weight of said solid dispersion.

28. The composition of claim 26 comprising about 1 to about 50% by weight of said solid dispersion.

29. The composition of claim 26 comprising about 1 to about 30% by weight of said solid dispersion.

30. The composition of claim 26 comprising about 1 to about 20% by weight of said solid dispersion.

31. The composition of claim 26 comprising about 1 to about 10% by weight of said solid dispersion.

32. A dosage form comprising the solid dispersion of claim 1.

33. The dosage form of claim 32 wherein said dosage form is for oral, transdermal, or implantation administration.

34. The dosage form of claim 32 wherein said dosage form is a tablet or capsule.

35. A method of treating a mammal having a disease or syndrome associated with estrogen deficiency or excess of estrogen comprising administering to said mammal a therapeutically effective amount of the solid dispersion of claim 1.

36. A method of treating a mammal having a disease or disorder associated with proliferation or abnormal development of endometrial tissues comprising administering to said mammal a therapeutically effective amount of the solid dispersion of claim 1.

37. A method of lowering cholesterol in a mammal comprising administering to said mammal a therapeutically effective amount of the solid dispersion of claim 1.

38. A method of inhibiting bone loss in a mammal comprising administering to said mammal a therapeutically effective amount of the solid dispersion of claim 1.

39. A method of treating breast cancer in a mammal comprising administering to said mammal a therapeutically effective amount of the solid dispersion of claim 1.

40. A method of treating postmenopausal woman for one or more vasomotor disturbances comprising administering to said postmenopausal woman a therapeutically effective amount of the solid dispersion of claim 1.

41. The method of claim 40 wherein said vasomotor disturbance is hot flush.
